(19) United States
(12) Patent Application Publication
(10) Pub. No.: US 2025/0040826 A1
(43) Pub. Date: Feb. 6, 2025

(54) IMPROVED ELECTRICAL TOMOGRAPHY MEASUREMENT DEVICE

(71) Applicant: TOUCH SENSITY, Pessac (FR)

(72) Inventors: Philippe FREITAS, Pessac (FR); Ganna PUGACH, Pessac (FR); Charles PASSET, Pessac (FR)

(73) Assignee: TOUCH SENSITY, Pessac (FR)

(21) Appl. No.: 18/722,863

(22) PCT Filed: Dec. 20, 2022
(86) PCT No.: PCT/FR2022/052441

§ 371 Date: Jun. 21, 2024

(30) Foreign Application Priority Data
Dec. 24, 2021 (FR) FR2114482

(51) Int. Cl. A61B 5/0536; A61B 5/00

(52) U.S. Cl. CPC A61B 5/0536; A61B 5/7225

(57) ABSTRACT

An electrical tomography measurement device for a solid substrate includes a current or voltage source, electrodes connectable to a solid substrate to be measured and an analogue-to-digital converter comprising two measurement inputs and a reference input. The converter can output a digital signal corresponding to the difference between the voltage of the two measurement inputs proportional to a voltage level provided by the reference input. The current or voltage source can be controllably connected to a pair of electrodes, such that a current from the current or voltage source flows through the substrate to be measured in a chosen excitation sequence. The two measurement inputs of the analogue-to-digital converter can each be connected to a respective electrode of a pair of electrodes from the electrodes chosen according to the excitation sequence. The current or voltage source is connected to the reference input of the analogue-to-digital converter.

The invention relates to the field of electrical process tomography (EPT).

Electrical process tomography, which comprises electrical capacitance tomography (ECT), electrical impedance tomography (EIT) and electrical resistance tomography (ERT), is based on the specific properties of the materials primarily detected by each technique:
EIT is useful for a process which has a continuous conductive phase, and
electrical resistance tomography (ERT) is a particular case of EIT, when the real component of the electrical impedance is the dominant property of the materials,
ECT detects the distribution of the permittivity of the materials dispersed in a process with a barely conductive continuous phase.

EPT has known applications in fluid measurement, in particular in the case of industrial processes. In the case of EIT, the sensor consists of several electrodes arranged at the periphery of the inner wall of the tank of the process or of the pipe, in contact with the fluid of the process but without intrusion into the fluid. An alternating current is applied to some electrodes and voltages are measured from the other electrodes, according to a predefined detection strategy. Afterwards, these voltage measurements are used to reconstruct the impedance distribution inside the tank with a specific inverse algorithm.

In the case of a substrate to be measured that is solid (i.e. not liquid and not gaseous), developments are more rare. EIT applied to a solid substrate is based on the reconstruction of the electric field of a conductive portion of the substrate. This non-invasive technique is used in medical imaging to detect the internal bodies by applying the electrodes at the surface of the skin of a patient and by measuring the variations in the electric field.

Beside the medical field, EIT applied to a solid substrate has known some uses in the field of pressure detection. Thus, it has been used in the articles by Kato et al. (“Tactile sensor without wire and sensing element in the tactile region based on eit method”, IEEE Sensors, pages 792-795, 2007), and by Yao and Soleimani (“A pressure mapping imaging device based on electrical impedance tomography of conductive fabric”, Sensor Review, 32 (4): 310-317, 2012) to propose tactile sensors (pressure sensors). The articles by Nagakubo et al. (“A deformable and deformation sensitive tactile distribution sensor”, IEEE International Conference on Robotics and Biomimetics, ROBIO, pages 1301-1308, 2007), by Alirezaei et al. (“A highly stretchable tactile distribution sensor for smooth surfaced humanoids”, 7th IEEE-RAS International Conference on Humanoid Robots, pages 167-173, 2007 et al. “A tactile distribution sensor which enables stable measurement under high and dynamic stretch”, IEEE Symposium on 3D User Interfaces (3DUI), pages 87-93, 2009), and Tawil et al. (“Improved image reconstruction for an eit-based sensitive skin with multiple internal electrodes”, IEEE Transactions on Robotics, 27 (3): 425-435, 2011), have proposed “artificial skin” type tactile devices for robots. Their approach consists in injecting currents and in measuring voltages from electrodes connected on the edges of a conductive fabric, then applying the inverse problem analysis to reconstruct the local change in resistivity due to a pressure. Finally, the articles by Pugach et al. (“Electronic hardware design of a low cost tactile sensor device for physical human-robot interactions”, IEEE XXXIII International Scientific Conference Electronics and Nanotechnology, ELNANO, pages 445-449, 2013, “Neural learning of the topographic tactile sensory information of an artificial skin through a self-organising map”, Advanced Robotics, 29 (21): 1393-1409, 2015, and “Touch-based admittance control of a robotic arm using neural learning of an artificial skin”, in 2016 IEEE/RSJ International Conference on Intelligent Robots and Systems (IROS), pages 3374-3380, 2016) have described the use of neural networks to reconstruct the distribution of resistance within a conductive film and to locate pressure points.

Other applications have been made in the detection of structural defects in cement. Thus, the article by Milad Hallaji et al. “Electrical impedance tomography-based sensing skin for quantitative imaging of damage in concrete” 2014 Smart Mater. Struct. 23 085001 and the thesis by Kimmo Karhunen “Electrical resistance tomography imaging of concrete”, 2013, Publications of the University of Eastern Finland Dissertations in Forestry and Natural Sciences No. 122 show explorations of extensions of EIT to materials the measurement of the conductivity of which is more complicated than that of a tissue or skin, as well as the limits of such measurements.

Hence, EIT applied to solid substrates requires a conductive material allowing observing sufficiently large variations in impedance, as well as a good capacity to connect the electrodes for the measurement. In the case of materials with a low impedance variation or that are complicated to be connected to electrodes, EIT and EPT in general are proscribed, since it is too complex to obtain enough resolution to extract information from the measurement data.

The invention improves the situation. To this end, it provides an electrical process tomography measurement device for a solid substrate, comprising a current or voltage source, a plurality of electrodes adapted to be connected to a solid substrate to be measured and an analog-to-digital converter comprising two measurement inputs and a reference input, arranged to output a digital signal corresponding to the difference between the voltage of the two measurement inputs proportionally to a voltage level designated by the reference input, the current or voltage source can be connected to a pair of electrodes in the plurality of electrodes in a controlled manner, so that a current from the current or voltage source passes through said substrate to be measured according to a selected excitation sequence, each of the two measurement inputs of the analog-to-digital converter can be connected to a respective one of a pair of electrodes in the plurality of electrodes selected according to said excitation sequence, characterised in that the current or voltage source is connected to the reference input of the analog-to-digital converter.

This device is particularly advantageous because it allows using EPT and more particularly EIT for applications such as human-machine interface, non-destructive testing or for structure monitoring, including with materials that do not normally lend themselves to EPT or EIT.

Indeed, the use of the signal derived from the current or voltage source as a reference signal for the analog-to-digital converter allows cancelling out in the measurement the noise due to the signal of the stimulation itself. This allows increasing very considerably the resolution of the measurements and using EPT in contexts that have been inaccessible before.

According to various embodiments, the invention may have one or more of the following features:
the device further comprises a first multiplexer connected downstream of the current or voltage source and connected to the plurality of electrodes and a second multiplexer connected upstream of the current or voltage source and connected to the plurality of electrodes in order to carry out the excitation sequence,
the device further comprises a third multiplexer connected to one of the measurement inputs of the analog-to-digital converter and connected to the plurality of electrodes and a fourth multiplexer connected to the other one of the measurement inputs of the analog-to-digital converter and connected to the plurality of electrodes, allowing the selected connection to be made according to said excitation sequence,
the device further comprises a buffer arranged between the current or voltage source and the reference input of the analog-to-digital converter,
the device further comprises an operational amplifier whose output is connected to the reference input of the analog-to-digital converter and whose inputs are connected to a respective electrode of the pair of electrodes in the plurality of electrodes according to said excitation sequence,
the device further comprises a fifth multiplexer connected between the output of the first multiplexer and one of the inputs of the operational amplifier, and a sixth multiplexer connected between the output of the second multiplexer and the other input of the operational amplifier,
the current or voltage source is a direct-current one,
the current or voltage source is an alternating-current one,
the measurement device implements a human-machine interface measurement, and
the measurement device implements a structural health monitoring or non-destructive testing measurement.

Other features and advantages of the invention will appear more clearly upon reading the following description, derived from examples given for non-limiting illustrative purposes, derived from the drawings wherein:

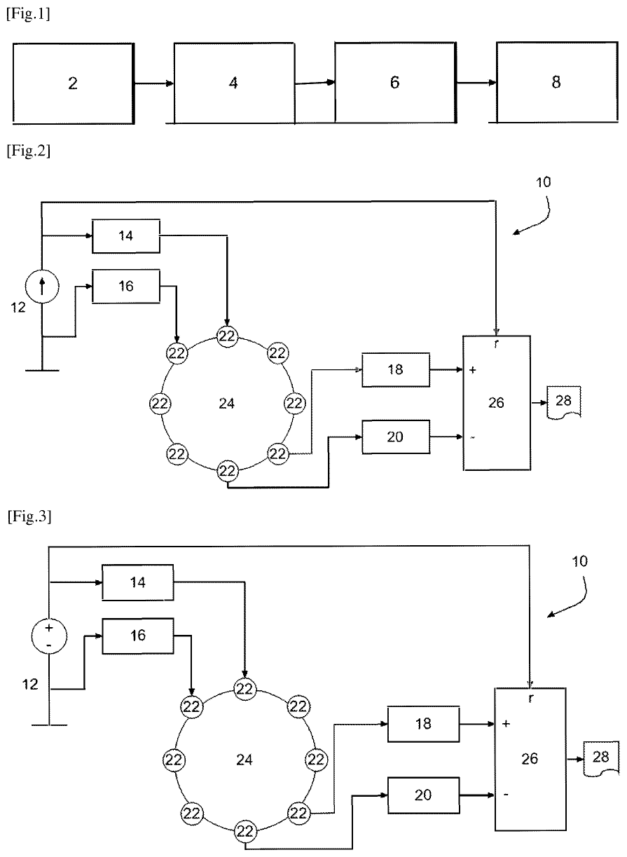

FIG. 1 shows a generic scheme for implementing an EPT device,

FIG. 2 shows a diagram of a first embodiment of a measuring device according to the invention,

FIG. 3 shows a diagram of a second embodiment of a measuring device according to the invention,

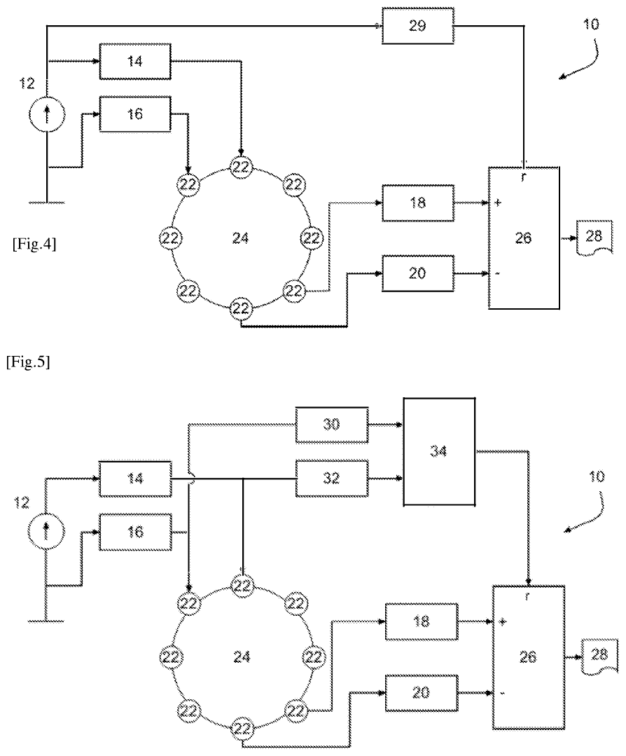

FIG. 4 shows a diagram of a third embodiment of a measuring device according to the invention,

FIG. 5 shows a diagram of a fourth embodiment of a measuring device according to the invention,

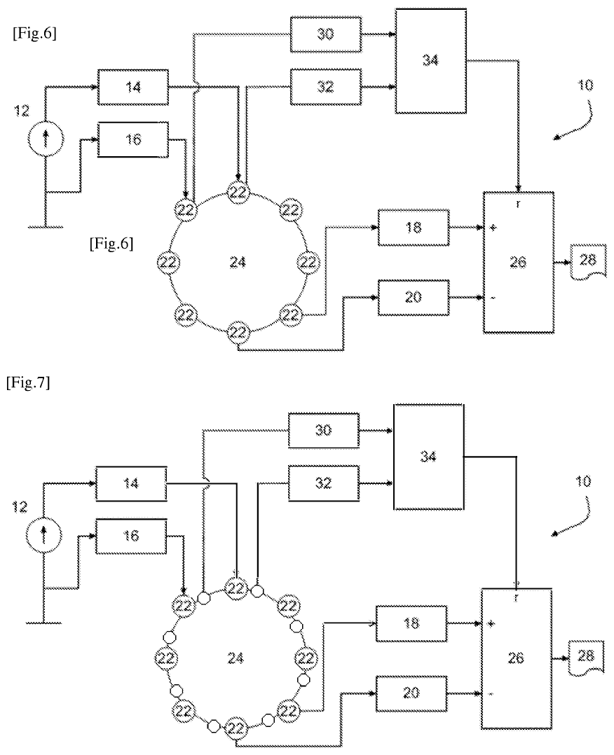

FIG. 6 shows a diagram of a fifth embodiment of a measuring device according to the invention, and

FIG. 7 shows a diagram of a sixth embodiment of a measuring device according to the invention.

The drawings and the description hereinafter essentially contain elements of a certain nature. Hence, they can not only serve to better understand the present invention, but also contribute to definition thereof, where appropriate.

The invention relates to EPT and EIT measurements used, for example, for monitoring the state of the SHM, NDT and HMI structures. These systems aim to monitor the variation of a specific physical element over time.

As shown in FIG. 1, these systems are generally composed of a stimulation subsystem 2, an element or substrate to be monitored 4, a data acquisition subsystem 6 integrating an analog-to-digital converter, and a data processing system 8 to determine the variation of the specific physical element over time. The data processing system 8 is not the main object of the invention. It is the whole allowing acquiring the measurements, up to the acquisition subsystem 6, which is so. Alternatively, the data acquisition subsystem 6 may comprise amplification and filtering operations prior to the analog-to-digital conversion.

The stimulation subsystem 2 is intended to generate a stimulation signal, the latter being an electrical one, and to apply the stimulation to the physical element or substrate 4 so that it responds with an electrical signal, the latter then being measured by the acquisition subsystem 6. Besides being of a different kind, i.e. voltage or current, the stimulation signal may be applied in the form of a constant value (DC) or of a value that is variable over time (AC), like a sinusoidal, square or pulsed wave, for example. The relationship between the measured signals and the stimulation signal is linear most of the time, which means that the measured response is proportional to the stimulation signal.

Any variation or any noise appearing within the system may mask the relevant information that the system aims to extract from the measured signal, mainly when the level of the measured signal is very low, less than a few microvolts, for example. Indeed, the data acquired by the system depend on a voltage or current source provided by the electronic system itself as a whole. This source is designed so as to be the most accurate, the most stable and the least noisy as possible, but it inherently contains variations and noise. Hence, these are injected directly into all of the dependent variables and consequently measured in return by the acquisition system.

The EIT measurement is based on a mechanism for measuring the electrical impedance at several points during which the stimulation source is successively conducted with different electrodes placed on the specimen or substrate, then multiple measurements are processed over all or part of the electrodes. In the simplest embodiment of such a system, the signals pass through multiplexers which select the electrodes to be stimulated and those to be measured. Finally, a data processing is used to plot the mapping of the impedance of the specimen. As mentioned hereinabove, this processing is not a central element of the invention.

The invention solves these problems by connecting the stimulation subsystem 2 and the data acquisition subsystem 6 the closest to the material. Thus, as shown in FIG. 2, an electrical process tomography measuring device 10 comprises a current source 12, multiplexers 14, 16, 18 and 20, eight electrodes 22 arranged on a substrate 24 to be subjected to measurement, and an analog-to-digital converter 26 which emits a digital measurement signal 28.

In the example described herein, the electrodes 22 are distributed in a substantially homogeneous manner over the substrate 24. The substantially circular shape is particularly suitable for reconstruction by the system 8. Alternatively, the device 10 can comprise fewer electrodes, for example 4, or more, for example 16 or more. In addition, the electrodes can be arranged in a non-homogeneous manner and according to other shapes than circularly depending on the applications.

The multiplexers 14 and 16 are respectively connected downstream and upstream of the current source 12, and reference may be connected to each of the electrodes 22. Thus, each pair of electrodes to which they are connected defines a stimulation circuit with the current source 12. Similarly, the multiplexers 18 and 20 are respectively connected to an input “+” and to an input “−” of the analog-to-digital converter 26. Thus, each pair of electrodes to which they are connected defines a circuit for measuring the stimulation, this measurement being transmitted to the terminals “+” and “−” of the analog-to-digital converter 26.

In the example described herein, the analog-to-digital converter 26 is part of a controller which is arranged to control the multiplexers 14, 16, 18 and 20. Alternatively, this control can be separated. Thus, the analog-to-digital converter 26 carries out the conversion into the digital form of the voltage measured at the terminals of the electrodes to which the multiplexers 16 and 18 are connected, with respect to the reference voltage at the input r of the analog-to-digital converter 26.

According to the invention, this input r is connected downstream of the current source 12, so that the latter receives substantially the same signal as the stimulation signal made with the multiplexers 14 and 16. Thus, the analog-to-digital converter 26 outputs a signal 28 which is free of any noise affecting the current source 12.

This approach is completely novel in the EPT field and more particularly in the EIT field. It should also be differentiated from equipment using conventional ratiometric measurement. Indeed, in these equipment, a strong and absolute reference physical element is used for the ratiometric measurement. This reference, different from the element to be monitored, is necessary to detect minimum changes in a system barely affected by noise. The methodology and the teaching of this approach are completely contrary to what is done in the invention, which provides for a stimulation of the element to be monitored and the use of the effect of the stimulation on the element itself as reference of the ratiometric measurement. On the other hand, the conventional ratiometric measurement applies to systems the connection of which to the element to be monitored is fixed over time. In contrast, the invention, using the element to be monitored as a reference, provides for broadening the principle of the ratiometric measurement to systems suited to EPT, within which the connection to the element to be monitored is done sequentially on a plurality of electrodes.

To carry out the measurements, the multiplexers 14, 16, 18 and 20 are excited sequentially according to an EIT excitation scheme. By “EIT excitation scheme”, it should be understood a scheme selected from among:
a neighbourhood scheme according to which the excitation current is introduced into neighbouring electrodes, and the voltage drop is measured successively in the other electrodes, each pair of electrodes being successively used to carry out an excitation,
an opposition scheme according to which the excitation current is introduced into diametrically-opposite electrodes, and the voltage is measured successively in the other electrodes, each pair of electrodes being successively used to carry out an excitation, and
a transverse scheme according to which the excitation current is introduced into opposite electrodes with respect to a fixed axis, and the voltage is measured successively in the other electrodes, each pair of electrodes being successively used to carry out an excitation.

Other excitation schemes can be considered.

In the example described herein, since the stimulation is carried out by means of a direct current source, the measurement voltage will be measured simultaneously in the electrodes. If this current source is an alternating-current one, the amplitude and the shift of the voltage would be measured with respect to the alternating current.

FIG. 3 shows a second embodiment, which will be described by its differences. According to this embodiment, the source 12 is herein a voltage source, which may also be a direct-current or alternating-current one.

This embodiment has the advantage of supplying a voltage directly identical at the input r and at the terminals of the multiplexers 14 and 16.

FIG. 4 shows a third embodiment. This embodiment is substantially close to the embodiment of FIG. 1, but the device 10 further includes a buffer 29. The buffer 29 is used herein in order to minimise the impact of the input r on the current source 12, in order to keep the latter as clean and stable as possible.

FIG. 5 shows a fourth embodiment. In this embodiment, in order to transmit to the input r a voltage that is as faithful as possible as that corresponding to the stimulation, multiplexers 30 and 32 are provided. These multiplexers are respectively connected at the output of the multiplexer 14 and the multiplexer 16, and are connected to the inputs of an operational amplifier 34 which thus guarantees that the voltage subjected to the input r is actually directly proportional to the stimulation voltage to which the substrate 24 is subjected. The multiplexers 30 and 32 are excited in correspondence with the multiplexers 16 and 14 respectively. This embodiment has the advantage of limiting the influence of voltage drops which may occur on the multiplexer 14 and the multiplexer 16 due to sending of a strong current to the substrate 24.

FIG. 6 shows a fifth embodiment. In this embodiment, the multiplexers 30 and 32 are directly connected to the electrodes 22 to which the multiplexers 16 and 14 are connected. This embodiment has the advantage of supplying at the input r a voltage integrating not only the noise generated by the multiplexers 14 and 16 but also that one due to the interactions between the multiplexers 14 and 16 and the substrate 24. Thus, the additional variations which are the noise and the voltage drops induced by the multiplexers 14 located between the current source 12 and the substrate 24 are injected into the reference voltage of the analog-to-digital converter 26.

Finally, FIG. 7 shows a sixth embodiment. In this embodiment, each of the electrodes 22 of the substrate 24 is duplicated by arranging an additional series of electrodes according to the same configuration as the electrodes 22, but with a slight offset with respect to the electrodes 22. Thus, each electrode 22 is associated with an electrode of the additional series of electrodes. In this embodiment, the multiplexers 30 and 32 are connected to the electrodes associated with the electrodes 22 to which the multiplexers 16 and 14 are connected. This embodiment has the advantage of supplying the input r with an actual voltage viewed from the substrate 24. This embodiment eliminates, by its nature, the noise generated by passage of the stimulation signal throughout the multiplexers 14 and 16, between the multiplexers 14 and 16 and the substrate 24 and finally throughout the contact resistances between the electrodes 22 and the substrate 24.

In the foregoing, it should be understood that the substrate 24 may be a human-machine interface or a substrate on which it is desired to perform NDT or a substrate whose structural health is to be monitored. Thus, although the illustrated examples relate to an EIT-type application, the device 10 finds application:
to any human-machine interface HMI intended to stimulate the physical interface and to measure in return the correlated signals resulting from the stimulation of the physical interface in order to measure any variation in local impedance inside the interface, and comprising a continuous or variable electrical stimulation source and an analog-to-digital converter,
to any HMI based on electrical impedance tomography and intended to stimulate the physical interface and to measure the correlated signals resulting from the stimulation of the physical interface in order to measure any variation in local impedance within the interface, and comprising a continuous or variable electrical stimulation source and an analog-to-digital converter,
to any HMI based on electrical resistance tomography and intended to stimulate the physical interface and to measure in return the correlated signals resulting from the stimulation of the physical interface in order to measure any variation in local impedance in the interface, and comprising a continuous or variable electrical stimulation source and an analog-to-digital converter,
to any HMI based on electrical capacitance tomography and intended to stimulate the physical interface and to measure in return the correlated signals resulting from the stimulation of the physical interface in order to measure any variation in local impedance within the interface, and comprising a variable electrical stimulation source and an analog-to-digital converter,
to any structural health monitoring (SHM) system intended to stimulate a physical specimen and to measure in return the correlated signals resulting from the stimulation of the physical component in order to measure any variation in local impedance inside the sample, and comprising a continuous or variable electrical stimulation source and an analog-to-digital converter,
to any SHM system based on electrical impedance tomography and intended to stimulate a physical specimen and to measure in return the correlated signals resulting from the stimulation of the physical component in order to measure any variation in local impedance inside the specimen, and comprising a continuous or variable electrical stimulation source and an analog-to-digital converter,
to any SHM system based on electrical resistance tomography and intended to stimulate a physical specimen and to measure in return the correlated signals resulting from the stimulation of the physical element in order to measure any variation in local impedance inside the specimen, and comprising a continuous or variable electrical stimulation source and an analog-to-digital converter,
to any SHM system based on electrical capacitance tomography and intended to stimulate a physical specimen and to measure in return the correlated signals resulting from the stimulation of the physical element in order to measure any variation in local impedance inside the specimen, and comprising a variable electrical stimulation source and an analog-to-digital converter,
to any non-destructive testing (NDT) system intended to stimulate a physical specimen and to measure in return the correlated signals resulting from the stimulation of the physical component in order to measure any variation in local impedance inside of the sample, and comprising a continuous or variable electrical stimulation source and an analog-to-digital converter,
to any NDT system based on electrical impedance tomography and intended to stimulate a physical specimen and to measure in return the correlated signals resulting from the stimulation of the physical component in order to measure any variation in local impedance inside the specimen, and comprising a continuous or variable electrical stimulation source and an analog-to-digital converter,
to any NDT system based on electrical resistance tomography and intended to stimulate a physical specimen and to measure in return the correlated signals resulting from the stimulation of the physical element in order to measure any variation in local impedance inside the specimen, and comprising a continuous or variable electrical stimulation source and an analog-to-digital converter, and
to any NDT system based on electrical capacitance tomography and intended to stimulate a physical specimen and to measure in return the correlated signals resulting from the stimulation of the physical element in order to measure any variation in local impedance inside the specimen, and comprising a variable electrical stimulation source and an analog-to-digital converter.

1. An electrical process tomography measurement device for a solid substrate, comprising:
a current or voltage source;
a plurality of electrodes configured to be connected to a solid substrate to be measured; and
an analog-to-digital converter comprising two measurement inputs and a reference input, configured to output a digital signal corresponding to a difference between the voltage of the two measurement inputs proportionally to a voltage level designated by the reference input,
the current or voltage source being configured to be connected to a pair of electrodes in the plurality of electrodes in a controlled manner, so that a current from the current or voltage source passes through said substrate to be measured according to a selected excitation sequence, each of the two measurement inputs of the analog-to-digital converter being configured to be connected to a respective one of a pair of electrodes in the plurality of electrodes selected according to said excitation sequence,
wherein the current or voltage source is connected to the reference input of the analog-to-digital converter,
the electrical process tomography measurement device further comprising:
a first multiplexer connected downstream of the current or voltage source and connected to the plurality of electrodes;
a second multiplexer connected upstream of the current or voltage source and connected to the plurality of electrodes in order to carry out the excitation sequence;
an operational amplifier whose output is connected to the reference input of the analog-to-digital converter and whose inputs are connected to a respective electrode of the pair of electrodes in the plurality of electrodes according to said excitation sequence;
a third multiplexer connected between the output of the first multiplexer and one of the inputs of the operational amplifier; and
a fourth multiplexer connected between the output of the second multiplexer and the other input of the operational amplifier.

2. The electrical process tomography measurement device according to claim 1, further comprising a fifth multiplexer connected to one of the measurement inputs of the analog-to-digital converter and connected to the plurality of electrodes, and a sixth multiplexer connected to the other one of the measurement inputs of the analog-to-digital converter and connected to the plurality of electrodes, allowing the selected connection to be made according to said excitation sequence.

3. The electrical process tomography measurement device according to claim 1, further comprising a buffer arranged between the current or voltage source and the reference input of the analog-to-digital converter.

4. The electrical process tomography measurement device according to claim 1, wherein the current or voltage source is a direct-current current or voltage source.

5. The electrical process tomography measurement device according to claim 1, wherein the current or voltage source is an alternating-current current or voltage source.

6. The electrical process tomography measurement device according to claim 1, wherein the measurement device implements a human-machine interface measurement.

7. The electrical process tomography measurement device according to claim 1, wherein the electrical process tomography measurement device implements a structural health monitoring or non-destructive testing measurement.
